(19) United States
(12) Patent Application Publication
(10) Pub. No.: US 2018/0180869 A1
(43) Pub. Date: Jun. 28, 2018

(54) OPTICAL MODULE AND ENDOSCOPE

(71) Applicant: OLYMPUS CORPORATION, Tokyo (JP)

(72) Inventor: Takahide MIYAWAKI, Kamiina-gun (JP)

(73) Assignee: OLYMPUS CORPORATION, Tokyo (JP)

(21) Appl. No.: 15/841,481

(22) Filed: Dec. 14, 2017

(30) Foreign Application Priority Data
Dec. 22, 2016 (JP) 2016-249115

(51) Int. Cl. G02B 23/24; G02B 23/26; H04N 5/225; A61B 1/00; A61B 1/05; A61B 1/07

(52) U.S. Cl. CPC G02B 23/2476; G02B 23/2484; G02B 23/26; H04N 2005/2255; A61B 1/00117; A61B 1/051; A61B 1/07; H04N 5/2256

(57) ABSTRACT

An optical module includes: an optical element; an optical fiber on which a first metal film is disposed; a wiring board which includes a through hole in which the optical fiber is inserted, wherein a second metal film is disposed on a wall surface of the through hole, and the second metal film is provided so as to annularly extend in a periphery of an opening of the through hole; and solder which fixes the optical fiber to the wiring board, and the solder is filled between the wall surface of the through hole and the outer peripheral face of the optical fiber and further forms a fillet covering a periphery of the opening of the wiring board and the outer peripheral face of the optical fiber.

### CROSS REFERENCE TO RELATED APPLICATION

This application is a continuation application of Japanese Patent Application No. 2016-249115 filed on Dec. 22, 2016, the entire contents of which are incorporated herein by this reference.

### BACKGROUND OF THE INVENTION

### 1. Field of the Invention

The present invention relates to an optical module including an optical element, an optical fiber and a holding member including a through hole in which the optical fiber is inserted, and an endoscope including the optical module.

### 2. Description of the Related Art

Endoscopes each include an image pickup device such as a CCD at a distal end portion of an elongated insertion portion. In recent years, use of an image pickup device having a large number of pixels for an endoscope has been considered. If an image pickup device having a large number of pixels is used, an amount of signal transmitted from the image pickup device to a signal processing apparatus (processor) becomes large, and thus, instead of electric signal transmission of an electric signal via a metal wiring, optical signal transmission of an optical signal via a thin optical fiber is preferable. For optical signal transmission, an E/O optical module (electrical/optical converter) configured to convert an electric signal to an optical signal and an O/E optical module (optical/electrical converter) configured to convert an optical signal to an electric signal are used.

Accurate positioning and fixing of an optical element of an optical module and an optical fiber configured to transmit an optical signal are important for efficient optical coupling. For accurate and easy positioning of the optical element and the optical fiber, a holding member (ferrule) including a through hole is used in the optical module. Upon insertion of the optical fiber to the through hole of the holding member, the optical element and the optical fiber are positioned.

Japanese Patent Application Laid-Open Publication No. 8-220347 discloses an optically airtight adapter in which an optical fiber coated with a metal film is inserted to a through hole of a flange and fixed via solder. For example, at an opening of the through hole having an inner diameter of 0.6 mm, a countersink having a diameter of 1 mm is formed. A nickel plating film is formed on the optical fiber having a clad diameter of 125 μm, and an outer diameter of the optical fiber thus becomes 550 μm. Then, the optical fiber is inserted to the through hole and fixed via solder.

In the optically airtight adapter, in order to seal the through hole, each of opposite ends of the optical fiber is fixed by the solder in the countersink, and in optically airtight adapters, a positional relationship between a center axis of a through hole and a center axis of an optical fiber is not important unlike optical modules.

Also, Japanese Patent Application Laid-Open Publication No. 7-168058 discloses an optical module formed in such a manner that when an optical element is mounted on a substrate, the optical element is self-aligned using solder bonding. Interfacial tension generated when the solder is melted allows the optical element to be mounted at a predetermined position without accurate positioning.

### SUMMARY OF THE INVENTION

An optical module according to an embodiment includes: an optical element in which an optical element section configured to emit or receive an optical signal and an external electrode connected to the optical element section are disposed in a front face; an optical fiber configured to transmit the optical signal, on an outer peripheral face of a distal end portion of which a first metal film is disposed; a holding member which includes a through hole in which the optical fiber is inserted, a second metal film is disposed on a wall surface of the through hole, and the second metal film is provided so as to annularly extend in a periphery of an opening of the through hole; and solder which fixes the optical fiber to the holding member, and the solder is filled between the wall surface of the through hole and the outer peripheral face of the optical fiber and further forms a fillet covering a periphery of the opening of the holding member and the outer peripheral face of the optical fiber.

An endoscope according to another embodiment includes an optical module and an image pickup device, and an electric signal outputted by the image pickup device or an electric signal inputted to the image pickup device is converted to an optical signal via the optical element, the optical module including: an optical element in which an optical element section configured to emit or receive an optical signal and an external electrode connected to the optical element section are disposed in a front face; an optical fiber configured to transmit the optical signal, on an outer peripheral face of a distal end portion of which a first metal film is disposed; a holding member which includes a through hole in which the optical fiber is inserted, a second metal film is disposed on a wall surface of the through hole, and the second metal film is provided so as to annularly extend in a periphery of an opening of the through hole; and solder which fixes the optical fiber to the holding member, and the solder is filled between the wall surface of the through hole and the outer peripheral face of the optical fiber and further forms a fillet covering a periphery of the opening of the holding member and the outer peripheral face of the optical fiber.

### BRIEF DESCRIPTION OF THE DRAWINGS

FIG. 1 is a cross-sectional view of an optical module according to a first embodiment;

FIG. 2 is a perspective view of the optical module according to the first embodiment;

FIG. 3 is an exploded view of the optical module according to the first embodiment;

FIG. 4 is a cross-sectional view of an optical module according to modification 1 of the first embodiment;

FIG. 5 is a cross-sectional view of an optical module according to modification 2 of the first embodiment;

FIG. 6 is a cross-sectional view of an optical module according to a second embodiment;

FIG. 7 is a perspective view of the optical module according to the second embodiment;

FIG. 8 is a cross-sectional view of an optical module according to modification 1 of the second embodiment;

FIG. 9 is a perspective view of the optical module according to modification 1 of the second embodiment; and

FIG. 10 is a perspective view of an endoscope according to a third embodiment.

### DETAILED DESCRIPTION OF THE PREFERRED EMBODIMENTS

### First Embodiment

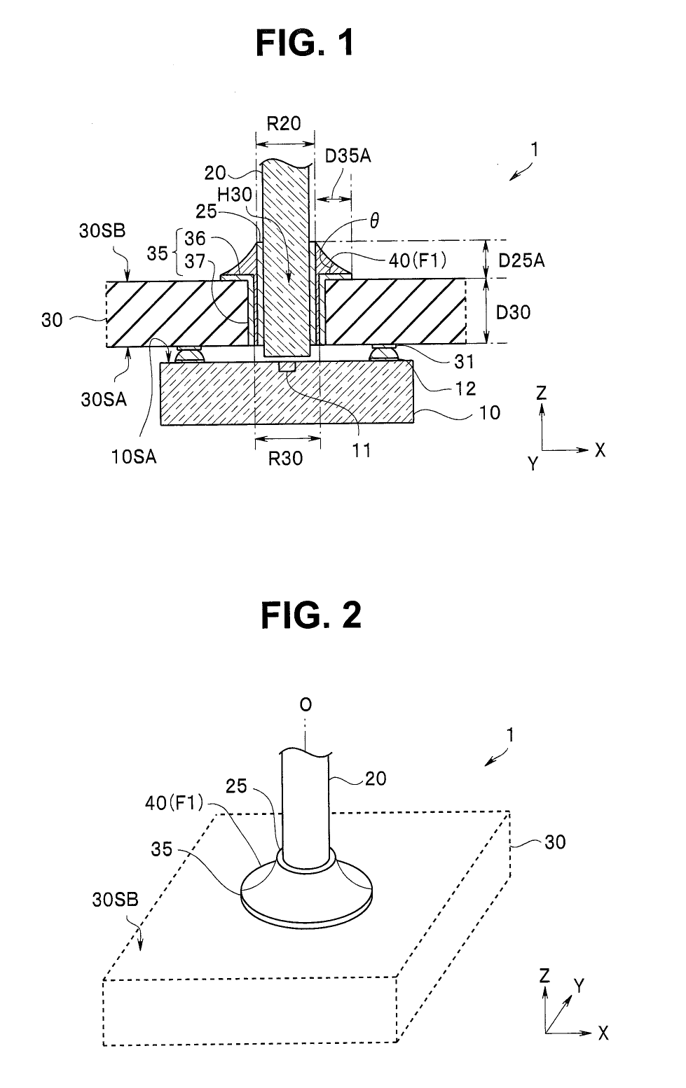

As illustrated in FIGS. 1 to 3, an optical module 1 according to the present embodiment includes an optical element 10, an optical fiber 20, a wiring board 30, and solder 40 fixing the optical fiber 20 to the wiring board 30.

It should be noted that in the below description, the drawings based on the respective embodiments are schematic ones and, e.g., a relationship between a thickness and a width of each part, and thickness ratios and relative angles among the respective parts are different from actual ones, and parts that are different in dimensional relationship or ratio depending on the drawings may be included in the drawings. Also, illustration of some of the components may be omitted. Also, “rear side” refers to a direction in which an optical fiber extends, that is, a direction in which a value of a Z-axis increases in, e.g., FIG. 1.

The optical element 10 is a surface-emitting laser chip (light-emitting optical element) including a light-emitting section 11, which is an optical element section configured to emit an optical signal, formed in a light-emitting surface 10SA, which is a front face. The optical element 10 having an ultracompact size, for example, dimensions of 250×300 μm in plan view, includes the light-emitting section 11 having a diameter of 20 μm and external electrodes 12 including bumps configured to supply a drive signal to the light-emitting section 11, in the light-emitting surface 10SA.

The optical fiber 20 includes, for example, a 50 μm-diameter core portion configured to transmit light, and a 125 μm-diameter clad portion covering an outer peripheral face of the core portion. The core portion includes glass having a refractive index that is slightly, for example, around 0.2% to 0.3%, larger than a refractive index of the clad portion.

The wiring board 30 including a first principal surface 30SA and a second principal surface 30SB is also a holding member including a through hole H30 in which the optical fiber 20 is inserted, in the optical module 1.

For a base of the wiring board 30, e.g., an FPC substrate, a glass epoxy substrate, a glass substrate or a silicon substrate is used.

Note that the wiring board 30 may include, e.g., a processing circuit for converting an electric signal to a drive signal for the optical element 10, and also, electronic components such as a chip capacitor and a drive IC may also be mounted on or incorporated in the wiring board 30.

Bonding electrodes 31 disposed on the first principal surface 30SA of the wiring board 30 are bonded to the external electrodes 12 of the optical element 10. In other words, the optical element 10 is flip-chip bonded to the wiring board 30 in such a manner that the light-emitting section 11 is arranged at a position at which the light-emitting section 11 faces the through hole H30. For example, the stud gold bumps of the external electrodes 12 and the bonding electrodes 31 of the wiring board 30 are ultrasonically bonded to each other.

The bonding part between the external electrodes 12 of the optical element 10 and the bonding electrodes 31 of the wiring board 30 may be sealed by a sealing resin having excellent humidity resistance and insulation property such as an epoxy resin or a silicone resin.

Note that it is possible that e.g., solder paste is printed on the bonding electrodes 31 of the wiring board 30 to form bumps, the optical element 10 is arranged at a predetermined position and then the solder is melted by means of, e.g., a reflow process to mount the optical element on the wiring board 30. In this case, a self-alignment effect allows the optical element 10 to be certainly mounted accurately at a desired position.

In the optical module 1, the optical fiber 20 inserted in the through hole 1130 of the wiring board 30 is fixed by the solder 40.

A first metal film 25 is disposed on an outer peripheral face, except an end face, of a distal end portion of the optical fiber 20. Also, a metal film 37 is disposed on a wall surface of the through hole H30 of the wiring board 30. The metal film 37 configures a second metal film 35 jointly with a metal film 36, which is an annular extension part provided so as to annularly extend in the periphery of an opening of the through hole H30.

Each of the first metal film 25 and the second metal film 35 includes a metal having good solder wettability, for example, copper, nickel or gold. The first metal film 25 is formed on a range of a length D25 of the distal end portion of the optical fiber 20. Then, upon insertion of the first metal film 25 to the through hole 1130, a part of the first metal film 25, the part having a length D25A, projects from the second principal surface 30SB of the wiring board 30.

On the other hand, the second metal film 35 is provided so as to extend from the opening of the through hole 1130 to a range of a length D35A. In other words, where R30 is a diameter of the through hole 1130, an outer diameter of the metal film 36 provided so as to extend annually is (R30+D35A+D35A).

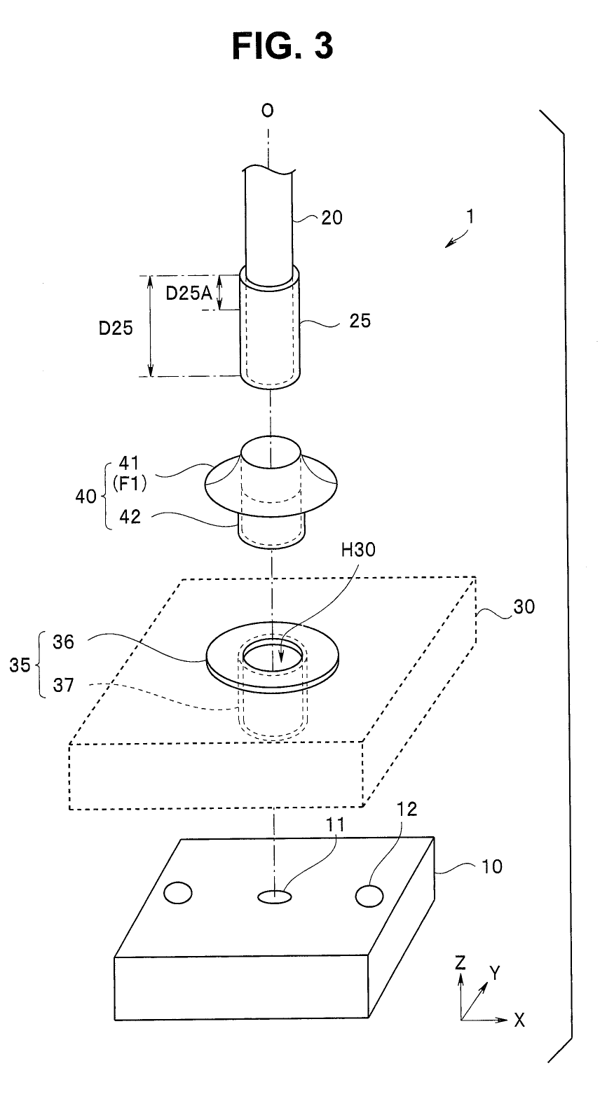

The solder 40 contains an Sn—Ag alloy having a melting point of 220° C. as a main component. As illustrated in FIG. 3, the solder 40 includes solder 42 filled in a space between the wall surface of the through hole H30 and the outer peripheral face of the optical fiber 20, and solder 41 forming a fillet F1 covering the periphery of the opening and the outer peripheral face of the optical fiber 20.

In other words, the solder 42 is filled in a space between the metal film 37 of the second metal film 35 disposed on the wall surface of the through hole H30 and the first metal film 25 disposed on the outer peripheral face of the optical fiber 20. The solder 42 preferably is uniformly filled in the space with no gap, but may include some voids. On the other hand, the solder 41 forms the fillet F1 including an outer face connecting an outer edge of the metal film 36 provided so as to annularly extend in the periphery of the opening of the through hole H30 and an outer edge on the rear side of the first metal film 25.

A fillet angle θ of the fillet F1 of the solder 41, that is, an angle θ formed by the second principal surface 30SB of the wiring board 30 and the outer face of the fillet F1 is 40 degrees. Note that, since the fillet F1 has an arced, fanned-out shape, the fillet angle θ is an angle at an intermediate point between the outer edge of the metal film 36 and the outer edge on the rear side of the first metal film 25.

In the optical module 1, the fillet angle θ is determined by the length D25A of the projection of the first metal film 25 from the second principal surface 30SB of the wiring board 30 and the length D35A of the metal film 36, which is the annular extension part. In other words, tan θ=(D25A/D35A).

In the optical module 1, bonding processing is performed using the solder 40, with the distal end portion of the optical fiber 20 inserted in the through hole H30 of the wiring board 30. A self-alignment effect provided by interfacial tension of the melted solder 40 allows a center axis of the optical fiber 20 to be arranged on a center axis of the through hole H30 and also allows a longitudinal direction of the optical fiber 20 to be arranged in parallel to the center axis of the through hole. In other words, interfacial tension of the solder 41 forming the fillet F1 and the solder 42 inside the through hole H30 allow the optical fiber 20 to be automatically aligned with an optical axis O of the optical element 10.

Thus, the optical module 1 has good optical transmission efficiency and is easy to manufacture.

For self-alignment, the fillet angle θ is preferably no less than 30 degrees and no more than 45 degrees. In the optical module 1, which is to be disposed in a distal end portion of an endoscope, for reduction in diameter, the length D35A of the metal film 36, which is the annular extension part, is, for example, 250 to 300 μm. Where the length D35A is 300 μm, the projection length D25A of the first metal film 25 is preferably 173 (300×tan (30 degrees)) to 300 μm (300×tan (45 degrees)). Also, where the length D35A is 250 μm, the projection length D25A of the first metal film 25 is preferably 144 to 250 μm.

Furthermore, an outer diameter R20 of the optical fiber 20 including the first metal film 25 is preferably no less than 5 μm and no more than 20 μm larger than the diameter R30 of the through hole H30 including the second metal film 35. For example, where the outer diameter of the optical fiber 20 is 125 μm and the inner diameter of the through hole H30 is 150 μm, twice a sum of a thickness of the first metal film 25 and a thickness of the second metal film 35 is preferably no less than 5 μm and no more than 20 μm.

Also, a thickness D30 of the wiring board 30 is preferably no less than three times, particularly preferably five times, the outer diameter R20 of the optical fiber 20 including the first metal film 25. For example, where R20 is 150 μm, the thickness D30 is preferably no less than 450 μm, particularly preferably no less than 750 μm.

Note that each of the thickness of the first metal film 25 and the thickness of the second metal film 35 is preferably no less than 0.5 μm and no more than 25 μm, particularly preferably no less than 1.25 μm and no more than 5 μm. The thicknesses within such range ensures wettability and bonding strength even if the thicknesses are decreased by diffusion of the metals to the solder 40 and prevents an increase in outer dimensions.

Each of the first metal film 25 and the second metal film 35 may be a single-layer film or a multi-layer film including, for example, an underlayer of copper and an upper layer of gold. The first metal film 25 and the second metal film 35 may have a same configuration (material) or have different configurations. For example, it is possible that the first metal film 25 is a gold film formed by a sputtering method and the second metal film 35 is a copper film formed by a plating method.

Also, the solder is not limited to the solder 40 containing an Sn—Ag alloy as a main component and any metal may be employed as long as such metal has a low melting point of no more than 300° C.

The above description has been provided taking, e.g., an optical module including a light-emitting element 10 as the optical element 10 as an example. However, the optical module may be an O/E optical module in which an optical element is a light-receiving element including a light-receiving section such as a photo diode. In other words, any optical module including an optical element in which an optical element section configured to emit or receive an optical signal and external electrodes connected to the optical element section are disposed in a front face provides effects that are the same as the effects of the optical module 1.

### Modifications of First Embodiment

Optical modules 1A, 1B according to modifications of the first embodiment are similar to the optical module 1, and thus, components that are the same in function as components of the optical module 1 are provided with reference numerals that are the same as the reference numerals of the components of the optical module 1 and description of such components will be omitted.

### Modification 1 of First Embodiment

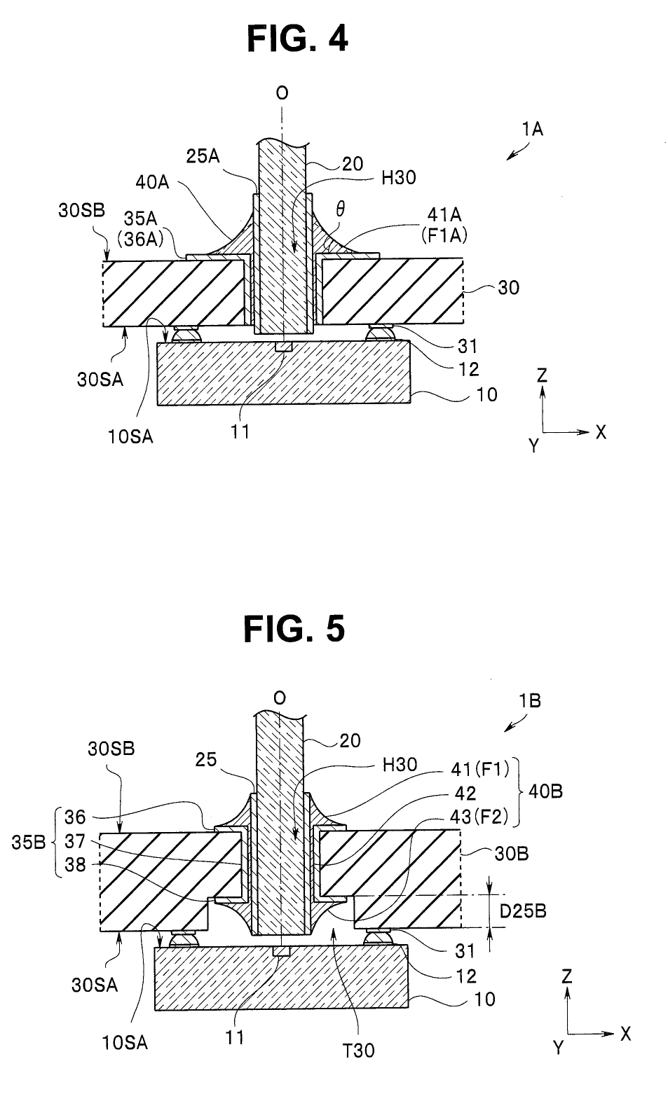

As illustrated in FIG. 4, in an optical module 1A according to the present modification, a length of a metal film 36A, which is an annular extension part, disposed on a second principal surface 30SB of a wiring board 30A and a length of a projection of a first metal film 25A from the second principal surface 30SB are larger than the lengths in the optical module 1. Thus, an outer face of a fillet F1A of solder 41A does not connect an outer edge of the metal film 36A and an outer edge on the rear side of the metal film 25A.

However, since solder 40A having a proper volume is used, a fillet angle θ is 35 degrees within a range of no less than 30 degrees and no more than 45 degrees.

In other word, an outer face of a fillet connecting an outer edge of a metal film 36 and an outer edge of a metal film 25 as in the optical module 1 is preferable because a fillet angle θ can easily be set to a predetermined angle. However, such requirement is not an essential requirement for the present invention.

### Modification 2 of First Embodiment

As illustrated in FIG. 5, in an optical module 1B according to the present modification, fillets F1, F2 are formed on a first principal surface 30SA and a second principal surface 30SB of a wiring board 30B, respectively. The first principal surface 30SA includes a recess portion T30 with a through hole H30 as a center, and the fillet F2 is formed on a bottom face of the recess portion T30.

In other words, solder 40B includes solder 41 forming the fillet F1 on the second principal surface 30SB, solder 42 inside the through hole H30 and solder 43 forming the fillet F2 on the first principal surface 30SA. As with the solder 41, an outer face of the solder 43 connects an outer edge on the front side of a first metal film 25, which projects from the first principal surface 30SA, and an outer edge of an annular extension part 38 of a second metal film 35B.

The fillet F2 configured by the solder 43 has a configuration that is substantially the same as a configuration of the fillet F1 configured by the solder 41. Then, the fillet F2 configured by the solder 43 is received in the recess portion T30. In other words, an inner diameter of the recess portion T30 only needs to be larger than an outer diameter of the annular extension part 38. Also, a sum of a depth of the recess portion T30 and a height of bumps of external electrodes 12 only needs to be equal to or larger than a projection length D25B of the first metal film 25.

In other words, if the height of the bumps of the external electrodes 12 is equal to or larger than the projection length D25B of the first metal film 25, the recess portion T30 is not necessary because space that can receive the fillet F2 is secured between a light-emitting surface 10SA of an optical element 10 and the first principal surface 30SA of the wiring board.

In the optical module 1B, a self-alignment effect provided by the fillet F2 (solder 43) enables easy alignment of the optical fiber 20 compared to the optical module 1.

### Second Embodiment

An optical module 2 according to a second embodiment is similar to the optical module 1, etc., and thus, components that the same in function as components of the optical module 1, etc., are provided with reference numerals that are the same as the reference numerals of the components of the optical module 1, etc., and description of such components will be omitted.

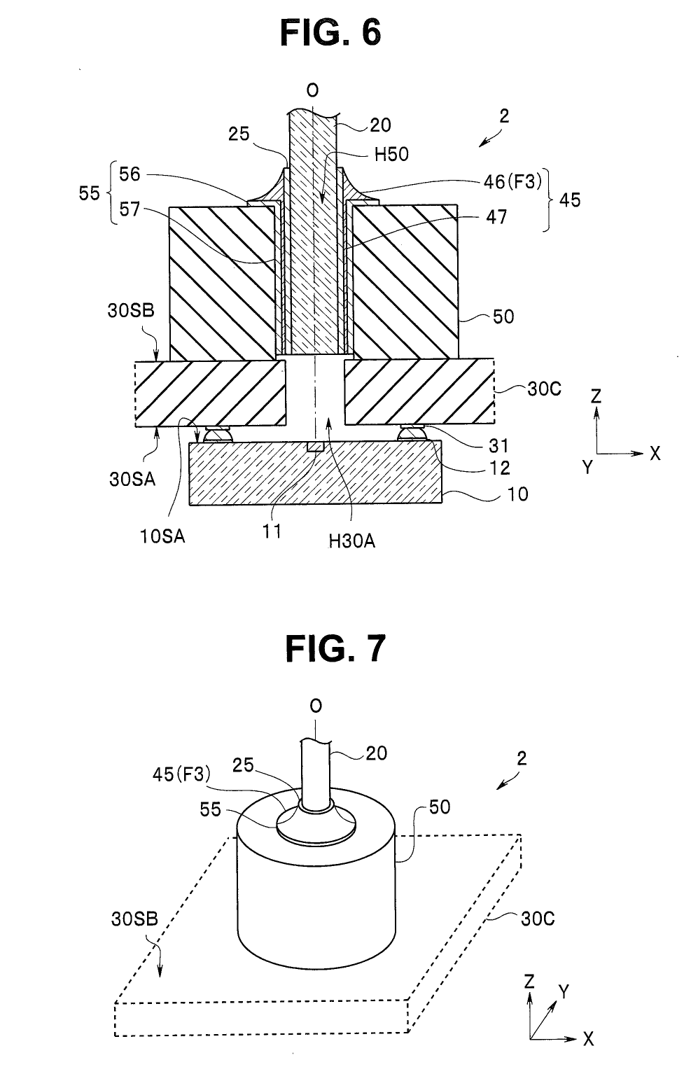

As illustrated in FIGS. 6 and 7, in the optical module 2, a holding member including a through hole H50 in which an optical fiber 20 is inserted is a cylindrical ferrule 50. Then, the optical module 2 includes a wiring board 30C including a third principal surface 30SA and a fourth principal surface 30SB, separately from the holding member (ferrule 50). An optical element 10 is mounted on the third principal surface 30SA of the wiring board 30C, and the ferrule 50 is disposed on the fourth principal surface 30SB.

The wiring board 30C includes a through hole H30A at a position at which the through hole H30A faces a light-emitting section 11. Then, the ferrule 50 is bonded to the fourth principal surface 30SB of the wiring board 30C via a non-illustrated bonding layer in a state in which the through hole H50 is arranged at a position where the through hole H50 faces the through hole H30A.

A length in an optical axis direction of the ferrule 50 is, for example, 700 μm, and a material of the ferrule 50 is, e.g., ceramic, silicon, glass or SUS. Note that the ferrule 50 may have, e.g., a conical shape or a polygonal prism shape. Also, an opening portion of the through hole H50 may be chamfered and thus have a tapered shape.

Solder 45 fixing the optical fiber 20 to the ferrule 50 includes solder 47 filled in a space between a wall surface of the through hole 1150 and an outer peripheral face of the optical fiber 20, and solder 46 forming a fillet F3 covering the periphery of the opening and the outer peripheral face of the optical fiber 20.

In other words, the solder 47 is filled in a space between a metal film 57 of a second metal film 55 disposed on the wall surface of the through hole H50 and a first metal film 25 disposed on the outer peripheral face of the optical fiber 20 (desirably, is uniformly filled in the space with no gaps). On the other hand, the solder 46 forms a fillet F3 including an outer face connecting an outer edge on the rear side of the first metal film 25 and an outer edge of a metal film 56 provided so as to annularly extend the periphery of the opening of the through hole H50.

Since a fillet angle of the fillet F3 configured by the solder 46 is no less than 30 degrees and no more than 45 degrees, the optical fiber 20 is automatically positioned on a center axis of the ferrule 50, that is, an optical axis O of the optical element 10 by a self-alignment effect provided by the melted solder 45.

Since the optical module 2 includes the ferrule 50, the optical module 2 can more stably hold the optical fiber 20 than the optical module 1, but has a larger length in the optical axis direction. In other words, the optical module 1 is shorter and smaller than the optical module 2, exclusive of the optical fiber 20.

### Modification of Second Embodiment

An optical module 2A according to a modification of the second embodiment is similar to the optical module 2, and thus, components that the same in function as components of the optical module 2 are provided with reference numerals that are the same as the reference numerals of the components of the optical module 2 and description of such components will be omitted.

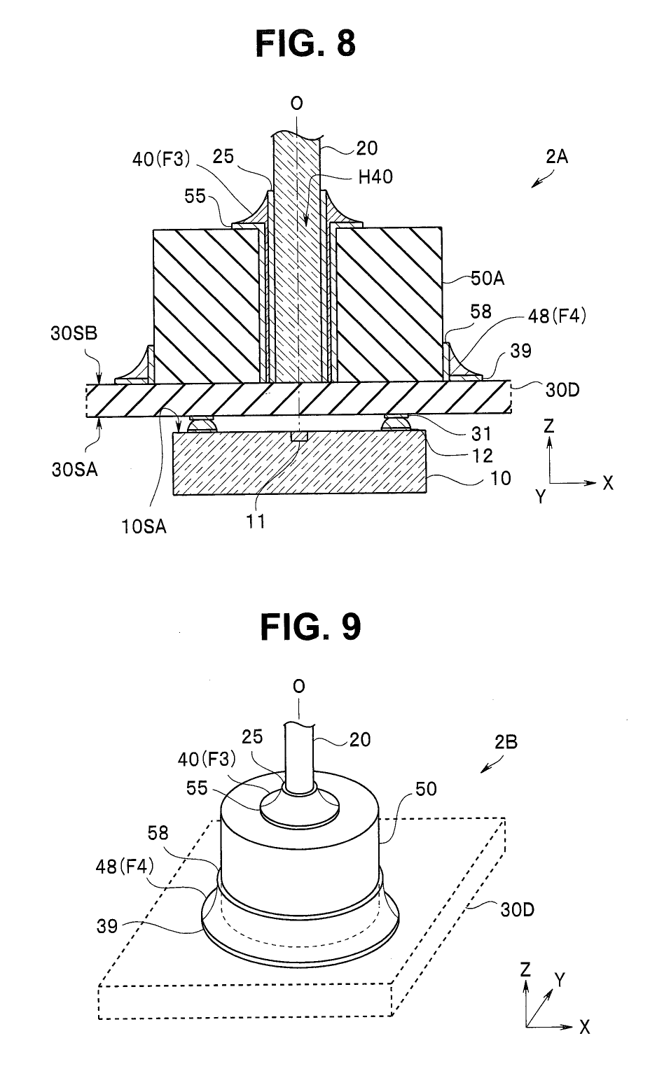

As illustrated in FIGS. 8 and 9, in the optical module 2A, a third metal film 58 is disposed on an outer peripheral face of a ferrule 50A, and a fourth metal film 39 is annularly disposed in the periphery of the ferrule 50A on a fourth principal surface 30SB of a wiring board 30D.

A first metal film 25, a second metal film 35, the third metal film 58 and the fourth metal film 39 may be the same or different in configuration (e.g., composition/stack structure), and also may be the same or different in thickness.

Note that the second metal film 35, the third metal film 58 and the fourth metal film 39 may form a continuous film. In other words, the entire outer peripheral face of the ferrule 50A may be covered by the metal film. Also, if the ferrule 50A includes a metal having a good solder wettability such as copper, no metal film is required. Disposing a solder mask pattern on an outer face of a metal ferrule to expose only a metal surface having a predetermined length (width) enables provision of effects that are the same as effects provided by a metal film 36 having a predetermined size. In other words, the metal exposed in the outer surface of the ferrule can be regarded as a metal film.

The ferrule 50A is fixed to the wiring board 30D by second solder 48. The second solder 48 forms a second fillet F4 connecting an outer edge of the third metal film 58 and an outer edge of the fourth metal film 39. A fillet angle θ of the second fillet F4 formed by the second solder 48 is 35 degrees within a preferable range of no less than 30 degrees and no more than 45 degrees.

If the optical fiber 20 is fixed to the ferrule 50A after the ferrule 50A is disposed on the wiring board 30D, for the second solder 48, solder having a composition having a melting point that is higher than a melting point of solder 40 is used.

In the optical module 2A, when the ferrule 50A is disposed on the wiring board 30D, also, a self-alignment effect provided by the melted second solder 48 enables the ferrule 50A to be accurately arranged at a predetermined position in the wiring board 30D, that is, a position at which an optical axis O of an optical element 10 and a center axis of a through hole H40 coincide with each other.

Note that, in the optical module 2A, the wiring board 30D is, for example, an optically-transparent substrate using an optically-transparent material including polyimide as a base. Thus, no through hole that serves as an optical path is formed. Optical transparency of a wiring board may be low for visible light wavelengths if the optical transparency is high for a wavelength of an optical signal of an optical element 10. For example, if an optical signal is infrared light, a wiring board using silicon as a base may be employed.

### Third Embodiment

An endoscope 9 according to a third embodiment includes the above-described optical module 1 (1A, 1B, 2 or 2A).

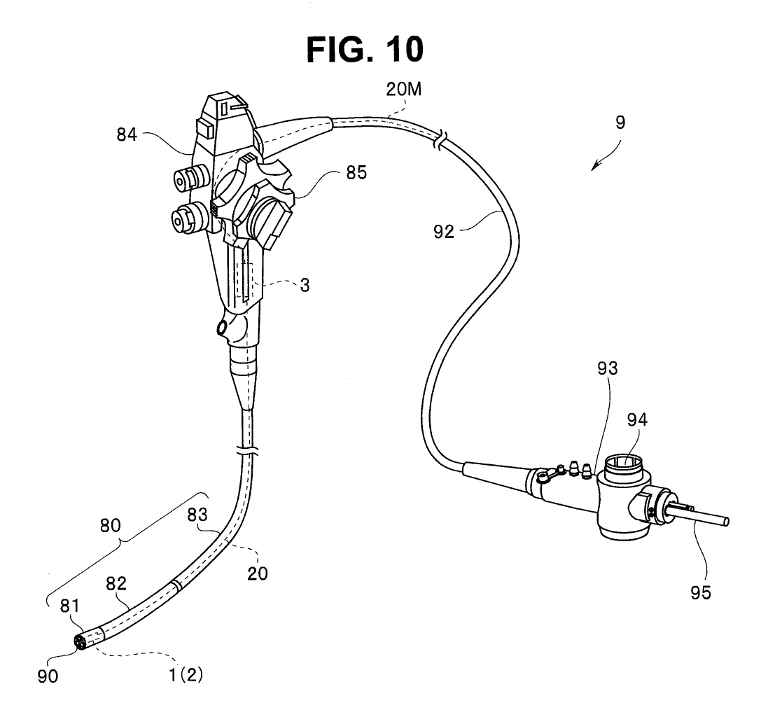

As illustrated in FIG. 10, the endoscope 9 according to the present embodiment includes an insertion portion 80, an operation portion 84 disposed on the proximal end side of the insertion portion 80, a universal cord 92 provided so as to extend from the operation portion 84, and a connector 93 provided on the proximal end side of the universal cord 92.

The insertion portion 80 includes a rigid distal end portion 81, a bending portion 82 for changing a direction of the distal end portion 81, and an elongated flexible portion 83, which are successively joined in this order.

In the distal end portion 81, e.g., an image pickup device 90 and the optical module 1, which is an E/O module configured to convert an image pickup signal (electric signal) from the image pickup device 90 to an optical signal, are disposed. The image pickup device 90 is, e.g., a CMOS (complementary metal oxide semiconductor) image sensor or a CCD (charge-coupled device).

In the operation portion 84, an angle knob 85 for operating the bending portion 82 is disposed, and an O/E module 3, which is an optical module configured to convert an optical signal to an electric signal, is disposed. The connector 93 includes an electric connector portion 94 connected to a processor (not illustrated) and a light guide connection portion 95 connected to a light source. The light guide connection portion 95 is connected to an optical fiber bundle configured to guide illuminating light to the rigid distal end portion 81. Note that the electric connector portion 94 and the light guide connection portion 95 of the connector 93 may be integrated.

In the endoscope 9, an image pickup signal is converted to an optical signal by, e.g., the optical module 1, which is an E/O module disposed in the distal end portion 81, and transmitted to the operation portion 84 via a thin optical fiber 20 inserted through the insertion portion 80. Then, the optical signal is converted again to an electric signal by the O/E module 3 disposed in the operation portion 84 and transmitted to the electric connector portion 94 via a metal wiring 20M inserted through the universal cord 92. In other words, inside the insertion portion 80, which has a small diameter, a signal is transmitted via the optical fiber 20, and inside the universal cord 92, which is not inserted to a body and is less limited in outer diameter, a signal is transmitted via the metal wiring 20M, which is thicker than the optical fiber 20.

Note that, if the O/E module 3 is arranged in the vicinity of the electric connector portion 94, the optical fiber 20 may be inserted through the universal cord 92 to the vicinity of the electric connector portion 94. Also, if the O/E module 3 is disposed in the processor, the optical fiber 20 may be inserted through the universal cord 92 to the connector 93.

Since in the endoscope 9, optical signal transmission of an optical signal via the thin optical fiber 20 is performed instead of electric signal transmission, the insertion portion 80 is thin and less invasive. Furthermore, the endoscope 9 includes, e.g., the optical module 1 having good optical transmission efficiency and thus can display a favorable image and can easily be manufactured.

Note that, although the endoscope 9 is a flexible endoscope, but may be a rigid endoscope. Also, the endoscope according to the embodiment may be a medical endoscope or an industrial endoscope.

The present invention is not limited only to the embodiments explained above and can be variously modified and carried out in a range not departing from the spirit of the invention.

What is claimed is:

1. An optical module comprising:
an optical element in which an optical element section configured to emit or receive an optical signal and an external electrode connected to the optical element section are disposed in a front face;
an optical fiber configured to transmit the optical signal, on an outer peripheral face of a distal end portion of which a first metal film is disposed;
a holding member which includes a through hole in which the optical fiber is inserted, wherein a second metal film is disposed on a wall surface of the through hole, and the second metal film is provided so as to annularly extend in a periphery of an opening of the through hole; and
solder which fixes the optical fiber to the holding member,
wherein the solder is filled between the wall surface of the through hole and the outer peripheral face of the optical fiber and further forms a fillet covering a periphery of the opening of the holding member and the outer peripheral face of the optical fiber.

2. The optical module according to claim 1, wherein the fillet includes an outer face connecting an outer edge of the first metal film and an outer edge of the annular extension part of the second metal film, and a fillet angle is no less than 30 degrees and no more than 45 degrees.

3. The optical module according to claim 2, wherein:
the holding member is a wiring board including a first principal surface and a second principal surface;
the optical element is mounted on the first principal surface; and
the fillet is forming on the second principal surface.

4. The optical module according to claim 3, wherein the fillet is formed on each of the first principal surface and the second principal surface.

5. The optical module according to claim 2, further comprising a wiring board including a third principal surface and a fourth principal surface,
wherein the optical element is mounted on the third principal surface and the holding member is disposed on the fourth principal surface.

6. The optical module according to claim 5, wherein:
a third metal film is disposed on an outer peripheral face of the holding member;
a fourth metal film is annularly disposed on a periphery of the holding member on the fourth principal surface of the wiring board; and
second solder fixing the holding member to the wiring board forms a second fillet connecting an outer edge of the third metal film and an outer edge of the fourth metal film.

7. An endoscope comprising:
the optical module according to claim 1; and
an image pickup device,
wherein an electric signal outputted by the image pickup device or an electric signal inputted to the image pickup device is converted to the optical signal by the optical element.
